(19) United States
(12) Patent Application Publication
(10) Pub. No.: US 2020/0181519 A1
(43) Pub. Date: Jun. 11, 2020

(54) LUBRICANT BASE OIL FOR FLUID BEARING

(71) Applicant: NEW JAPAN CHEMICAL CO., LTD., Kyoto-shi, Kyoto (JP)

(72) Inventors: Akinobu Takegami, Kyoto-shi (JP); Hirotsugu Mochida, Kyoto-shi (JP); Yumi Mandai, Kyoto-shi (JP)

(73) Assignee: NEW JAPAN CHEMICAL CO., LTD., Kyoto-shi, Kyoto (JP)

(21) Appl. No.: 16/642,107

(22) PCT Filed: Oct. 23, 2018
(86) PCT No.: PCT/JP2018/039260

§ 371 Date: Feb. 26, 2020

(30) Foreign Application Priority Data
Oct. 26, 2017 (JP) 2017-206968

(51) Int. Cl. C10M 105/34; C07C 67/60; C07C 69/24; C10M 169/04; C10M 129/10; C10M 133/12; F16C 33/10

(52) U.S. Cl. CPC C10M 105/34; C07C 67/60; C07C 69/24; C10M 169/04; C10M 129/10; C10M 133/12; C10N 2230/10; C10M 2207/023; C10M 2215/06; C10M 2207/2815; C10M 2290/00; C10N 2240/02; F16C 33/109

(57) ABSTRACT

An object of the present invention is to provide an ester-based lubricating base oil for a fluid bearing that has excellent hydrolysis resistance and low-temperature fluidity, a high viscosity index, and good evaporation resistance. The present invention relates to a lubricating base oil for a fluid bearing comprising a compound represented by general formula (1):

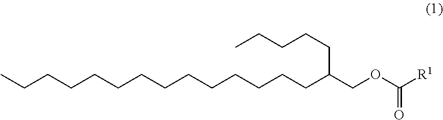

wherein R¹ represents a linear C₇-C₁₃ alkyl group, and a compound represented by general formula (2):

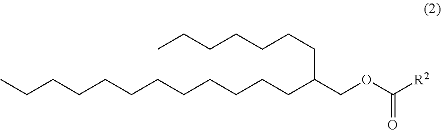

wherein R² represents a linear C₇-C₁₃ alkyl group; and relates to a base oil composition comprising the base oil.

### TECHNICAL FIELD

The present invention relates to a lubricating base oil for a fluid bearing.

### BACKGROUND ART

Ball bearings and roller bearings have been used as bearings in motors mounted in hard disk drives (HDDs) etc. However, due to demand for, for example, downsizing, vibration reduction, and decreased noise of motors, fluid bearings have recently been developed; and fluid dynamic bearings and oil-impregnated sintered bearings have been put into practical use as such fluid bearings.

A fluid dynamic bearing supports a rotating shaft by oil film pressure of a lubricating oil present in the gap between the outer peripheral surface of the shaft and the inner peripheral surface of the sleeve. In at least one of the outer peripheral surface of the shaft and the inner peripheral surface of the sleeve, dynamic-pressure grooves are provided, and the sliding surface of the rotating shaft is supported in a floating manner by a lubricating oil film formed by the dynamic-pressure effect. An oil-impregnated sintered bearing is a bearing in which a porous body made of sintered metal or the like is impregnated with a lubricating oil or a lubricating grease to impart a self-lubricating function.

Along with the higher performance of audiovisual equipment or office automation equipment, and the widespread use of cellular phones, spindle motors equipped with fluid bearings are being increasingly used. Due to the recent strong demand for higher speed and downsizing of spindle motors, fluid bearings are required to achieve lower torque. To meet the demand for lower torque, lubricating base oils with a relatively low viscosity have been selected. Examples of such low-viscosity lubricating base oils include synthetic hydrocarbon-based lubricating base oils such as poly-α-olefin; ester-based lubricating base oils such as aliphatic dibasic acid diester, neopentyl-type polyol ester, and fatty acid monoester; and the like. Lubricating base oils for fluid bearings that use such a base oil have been proposed (Patent Literature 1 to 8).

Among these, ester-based lubricating base oils, which have excellent viscosity properties, low-temperature fluidity, etc., are often used as lubricating base oils for fluid bearings.

However, since such an ester-based lubricating base oil contains an ester group in its molecular structure, hydrolysis occurs due to moisture, which may be problematic when a spindle motor is used for a long period of time.

Moreover, because the heads and disks of HDDs have become more advanced, the prevention of pollution due to outgassing etc. has been strongly demanded (Patent Literature 9). Although a mechanical structure that makes it difficult for a lubricating base oil to evaporate has been developed, and a device has been designed so that generated gas does not enter the disk or head, there is also a need to improve the evaporation resistance of the lubricating base oil for fluid bearings that is used.

### CITATION LIST

### Patent Literature

PTL 1: JPH11-514778A

PTL 2: JPH11-514779A

PTL 3: JP2000-500898A

PTL 4: JP2003-119482A

PTL 5: WO2004/018595

PTL 6: JP2004-084839A

PTL 7: JP2005-290256A

PTL 8: JP2008-007741A

PTL 9: JP2012-181888A

### SUMMARY OF INVENTION

### Technical Problem

An object of the present invention is to provide a lubricating base oil for a fluid bearing that has excellent hydrolytic stability and low-temperature fluidity; a high viscosity index; and good evaporation resistance.

### Solution to Problem

The present inventors conducted extensive research to achieve the above object, and found that a lubricating base oil comprising a specific linear aliphatic monocarboxylic acid (2-pentylhexadecyl) ester and a specific linear aliphatic monocarboxylic acid (2-heptyltetradecyl) ester is a lubricating base oil for a fluid bearing that is excellent in hydrolytic stability and low-temperature fluidity, and that has a high viscosity index and good evaporation resistance. Based on this finding, the present inventors accomplished the present invention.

Specifically, the present invention provides the following lubricating base oil for a fluid bearing.

1. A lubricating base oil for a fluid bearing, the lubricating base oil comprising a compound represented by general formula (1):

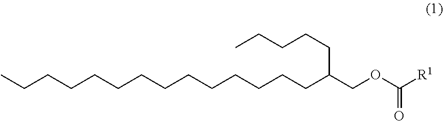

wherein R¹ represents a linear C₇-C₁₃ alkyl group; and a compound represented by general formula (2):

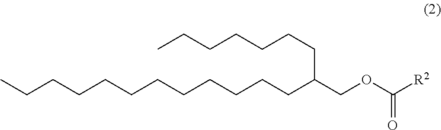

wherein R¹ represents a linear C₇-C₁₃ alkyl group.
2. The lubricating base oil for a fluid bearing according to Item 1, wherein R¹ in general formula (1) is a linear C₈-C₁₁ alkyl group, and R² in general formula (2) is a linear C₈-C₁₁ alkyl group.
3. The lubricating base oil for a fluid bearing according to Item 1 or 2, wherein R¹ in general formula (1) and R² in general formula (2) are the same linear C₈-C₁₁ alkyl group.
4. The lubricating base oil for a fluid bearing according to any one of Items 1 to 3, wherein the weight ratio of the compound represented by general formula (1) to the compound represented by general formula (2) is 20:80 to 70:30.
5. The lubricating base oil for a fluid bearing according to any one of Items 1 to 4, wherein the total amount of the compound represented by general formula (1) and the compound represented by general formula (2) in the lubricating base oil for a fluid bearing is 90 wt % or more.
6. The lubricating base oil for a fluid bearing according to any one of Items 1 to 5, wherein the lubricating base oil for a fluid bearing is a lubricating base oil for a fluid dynamic bearing or a lubricating base oil for an oil-impregnated sintered bearing.
7. A lubricating oil composition for a fluid bearing, the composition comprising the lubricating base oil for a fluid bearing according to any one of Items 1 to 6.
8. The lubricating oil composition for a fluid bearing according to Item 7, further comprising an antioxidant.
9. The lubricating oil composition for a fluid bearing according to Item 8, wherein the antioxidant is a phenolic antioxidant and/or an amine-based antioxidant.
10. A fluid bearing comprising the lubricating oil composition for a fluid bearing according to Item 7 or 8.
11. A spindle motor comprising the fluid bearing according to Item 10.
12. A method for producing a mixture (lubricating base oil for a fluid bearing) comprising a compound represented by general formula (1) and a compound represented by general formula (2), R¹ in general formula (1) and R² in general formula (2) being the same linear C₈-C₁₁ alkyl group, the method comprising:

(I) subjecting 1-tetradecanol and 1-heptanol to a dimerization reaction to obtain a crude dimerized alcohol product,

(II) (a) distilling the obtained crude dimerized alcohol product to separately collect 2-pentylhexadecanol and 2-heptyltetradecanol, and mixing the 2-pentylhexadecanol and the 2-heptyltetradecanol at a predetermined ratio, thereby obtaining a mixture comprising the 2-pentylhexadecanol and the 2-heptyltetradecanol, or (b) distilling the obtained crude dimerized alcohol product to remove a low-boiling fraction and a high-boiling fraction, thereby obtaining a mixture comprising 2-pentylhexadecanol and 2-heptyltetradecanol; and

(III) reacting the obtained mixture with a compound represented by general formula (1a):

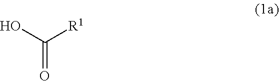

wherein R¹ is the same as above.
13. A method for producing a lubricating base oil for a fluid bearing, the method comprising mixing a compound represented by general formula (1) and a compound represented by general formula (2).

### Advantageous Effects of Invention

The lubricating base oil for a fluid bearing of the present invention is excellent in hydrolytic stability and low-temperature fluidity, and has a high viscosity index and good evaporation resistance.

### BRIEF DESCRIPTION OF DRAWINGS

FIG. 1 illustrates an example of a cross-sectional view of the fluid bearing of the present invention.

FIG. 2 illustrates an example of a cross-sectional view of the spindle motor of the present invention.

### DESCRIPTION OF REFERENCE NUMERALS

1 Shaft
2 Sleeve
3, 4 Radial dynamic pressure-generating grooves
5, 6 Thrust dynamic pressure-generating grooves
7 Thrust plate
8 Counter plate
9 Lubricating oil composition
10 Hub
11 Base
12 Stator coil
13 Rotor magnet

### DESCRIPTION OF EMBODIMENTS

### 1. Lubricating Base Oil for Fluid Bearing

The lubricating base oil for a fluid bearing of the present invention comprises a compound represented by the following general formula (1), and a compound represented by the following general formula (2).

The compound represented by general formula (1):

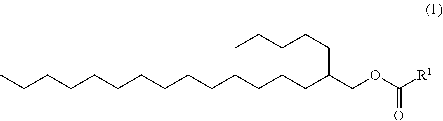

wherein R¹ represents a linear C₇-C₁₃ alkyl group can be produced by, for example, an esterification reaction of a compound represented by general formula (1a):

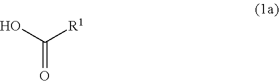

wherein R¹ is the same as above, with 2-pentylhexadecanol.

In the compound represented by general formula (1a), R¹ is a linear C₇-C₁₃ alkyl group, and particularly preferably a linear C₈-C₁₁ alkyl group. When R¹ contains fewer than 7 carbon atoms, the resulting base oil has a very low viscosity index, and the amount of evaporation increases. When R¹ contains more than 13 carbon atoms, the viscosity of the resulting base oil increases, and the low-temperature fluidity undesirably deteriorates. Specific examples of the compound represented by general formula (1a) include n-octanoic acid, n-nonanoic acid, n-decanoic acid, n-undecanoic acid, n-dodecanoic acid, n-tridecanoic acid, and n-tetradecanoic acid. Among these, n-nonanoic acid, n-decanoic acid, n-undecanoic acid, and n-dodecanoic acid are preferable.

Specific examples of the compound represented by general formula (1) include 2-pentylhexadecyl n-octanoate, 2-pentylhexadecyl n-nonanoate, 2-pentylhexadecyl n-decanoate, 2-pentylhexadecyl n-undecanoate, 2-pentylhexadecyl n-dodecanoate, 2-pentylhexadecyl n-tridecanoate, and 2-pentylhexadecyl n-tetradecanoate. Among these, 2-pentylhexadecyl n-nonanoate, 2-pentylhexadecyl n-decanoate, 2-pentylhexadecyl n-undecanoate, and 2-pentylhexadecyl n-dodecanoate are preferable.

The compound represented by general formula (2):

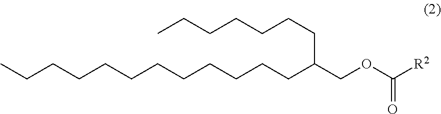

wherein R² represents a linear C₇-C₁₃ alkyl group can be produced by, for example, an esterification reaction of a compound represented by general formula (2a):

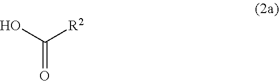

wherein R² is the same as above, with 2-heptyltetradecanol.

In the compound represented by general formula (2a), R² is a linear C₇-C₁₃ alkyl group, and particularly preferably a linear C₈-C₁₁ alkyl group. When R² contains fewer than 7 carbon atoms, the resulting base oil has a very low viscosity index, and the amount of evaporation increases. When R² contains more than 13 carbon atoms, the viscosity of the resulting base oil increases, and the low-temperature fluidity undesirably deteriorates. Specific examples of the compound represented by general formula (2a) include n-octanoic acid, n-nonanoic acid, n-decanoic acid, n-undecanoic acid, n-dodecanoic acid, n-tridecanoic acid, and n-tetradecanoic acid. Among these, n-nonanoic acid, n-decanoic acid, n-undecanoic acid, and n-dodecanoic acid are preferable.

Specific examples of the compound represented by general formula (2) include 2-heptyltetradecyl n-octanoate, 2-heptyltetradecyl n-nonanoate, 2-heptyltetradecyl n-decanoate, 2-heptyltetradecyl n-undecanoate, 2-heptyltetradecyl n-dodecanoate, 2-heptyltetradecyl n-tridecanoate, and 2-heptyltetradecyl n-tetradecanoate. Among these, 2-heptyltetradecyl n-nonanoate, 2-heptyltetradecyl n-decanoate, 2-heptyltetradecyl n-undecanoate, and 2-heptyltetradecyl n-dodecanoate are preferable.

The weight ratio of the compound represented by general formula (1) to the compound represented by general formula (2) is preferably 20:80 to 70:30, more preferably 40:60 to 60:40, and particularly preferably 45:55 to 55:45.

The total amount of the compound represented by general formula (1) and the compound represented by general formula (2) in the lubricating base oil for a fluid bearing is preferably 90 wt % or more, more preferably 95 wt % or more, and particularly preferably 98 wt % or more.

The lubricating base oil for a fluid bearing preferably has a kinematic viscosity at 40° C. of 8 mm²/s or more, and less than 20 mm²/s; more preferably 10 mm²/s or more, and less than 18 mm²/s; and particularly preferably 11 mm²/s or more, and less than 16 mm²/s. When the kinematic viscosity at 40° C. is 8 mm²/s or more, the lubrication performance is excellent. When the kinematic viscosity at 40° C. is less than 20 mm²/s, the energy loss is small. The kinematic viscosity is a value obtained by the method described in the Examples below.

The lubricating base oil for a fluid bearing preferably has a viscosity index of more than 145, and particularly preferably more than 155. The higher the viscosity index, the more excellent the viscosity-temperature characteristics. The viscosity index is a value obtained by the method described in the Examples below.

The low-temperature properties of the lubricating base oil for a fluid bearing can be evaluated, for example, by measuring the pour point in a low-temperature fluidity test. The pour point of the lubricating base oil is preferably −7.5° C. or less, and particularly preferably −12.5° C. or less. The lower the pour point, the more excellent the low-temperature fluidity. The lubricating base oil for a fluid bearing of the present invention is a mixture of the compound represented by general formula (1) and the compound represented by general formula (2), and thus has excellent low-temperature fluidity. The pour point is a value obtained in the low-temperature fluidity test described in the Examples below.

The evaporation resistance of the lubricating base oil for a fluid bearing can be evaluated, for example, by using as an index the temperature at which the weight is reduced by 5% measured by a TG-DTA device. The temperature at which the weight of the lubricating base oil for a fluid bearing is reduced by 5% is preferably 270° C. or more, and particularly preferably 275° C. or more. The higher the temperature at which the weight is reduced by 5%, the more excellent the evaporation resistance. The temperature at which the weight is reduced by 5% is a value obtained in the evaporation resistance test described in the Examples below.

The hydrolytic stability (hydrolysis resistance) of the lubricating base oil for a fluid bearing can be evaluated, for example, by the amount of increase in the acid value after a hydrolysis test. The amount of increase in the acid value of the lubricating base oil after a hydrolysis test is preferably 0.5 KOH mg/g or less, and particularly preferably 0.25 KOH mg/g or less. The smaller the amount of increase in the acid value after a hydrolysis test, the more excellent the hydrolytic stability. The amount of increase in the acid value after a hydrolysis test is a value obtained in the hydrolytic stability test described in the Examples below.

The lubricating base oil for a fluid bearing can be prepared by mixing the compound represented by general formula (1) and the compound represented by general formula (2) produced as described above.

Alternatively, a mixture (lubricating base oil for a fluid bearing) comprising the compound represented by general formula (1) and the compound represented by general formula (2), wherein R¹ in general formula (1) and R² in general formula (2) are the same linear C₈-C₁₁ alkyl group, can be obtained by a production method comprising:

(I) subjecting 1-tetradecanol and 1-heptanol to a dimerization reaction to obtain a crude dimerized alcohol product;
(II) (a) distilling the obtained crude dimerized alcohol product to separately collect 2-pentylhexadecanol and 2-heptyltetradecanol, and mixing the 2-pentylhexadecanol and the 2-heptyltetradecanol at a predetermined ratio, thereby obtaining a mixture comprising the 2-pentylhexadecanol and the 2-heptyltetradecanol, or (b) distilling the obtained crude dimerized alcohol product to remove a low-boiling fraction and a high-boiling fraction, thereby obtaining a mixture comprising 2-pentylhexadecanol and 2-heptyltetradecanol; and
(III) reacting the obtained mixture with a compound represented by general formula (1a).

In step (I), 1-tetradecanol and 1-heptanol are subjected to a dimerization reaction (Guerbet reaction) in the presence of a catalyst and a base to obtain a crude dimerized alcohol product. This reaction can be carried out by a known method (for example, JPS49-035308).

This reaction can be carried out in the presence or absence of a solvent. When a solvent is used, examples of solvents include aromatic hydrocarbons such as toluene and xylene; and the like. The amount of solvent is generally 5 to 30 parts by weight per 100 parts by weight of the total amount of 1-tetradecanol and 1-heptanol.

Examples of catalysts include transition metal-containing catalysts, such as a copper chromium catalyst and a copper zinc catalyst. The amount of catalyst is generally 0.01 to 0.5 parts by weight per 100 parts by weight of the total amount of 1-tetradecanol and 1-heptanol.

Examples of bases include alkali metal hydroxides (e.g., lithium hydroxide, sodium hydroxide, and potassium hydroxide), alkali metal alkoxides (e.g., sodium methoxide and potassium tert-butoxide), and the like. The base can be used in the form of an aqueous solution. The amount of base is generally 0.5 to 5 parts by weight per 100 parts by weight of the total amount of 1-tetradecanol and 1-heptanol.

The molar ratio of 1-tetradecanol to 1-heptanol used in the reaction is generally 40:60 to 60:40, and preferably 55:45 to 45:55.

This reaction can be generally carried out at 100 to 300° C. for 1 to 10 hours. After completion of the reaction, a post-treatment is performed by a known method to obtain a crude dimerized alcohol product.

In step (II), the crude dimerized alcohol product obtained in step (I) is subjected to distillation (in particular, rectification) by using a known method to separately collect 2-pentylhexadecanol and 2-heptyltetradecanol, and the 2-pentyihexadecanol and the 2-heptyltetradecanol are then mixed at a predetermined ratio according to required properties of the base oil to obtain a mixture comprising the 2-pentylhexadecanol and the 2-heptyltetradecanol. The mixing ratio of the 2-pentylhexadecanol to the 2-heptyltetradecanol is preferably 20:80 to 70:30, more preferably 40:60 to 60:40, and particularly preferably 45:55 to 55:45, in terms of the weight ratio.

Alternatively, in step (II), the crude dimerized alcohol product obtained in step (I) is subjected to distillation (in particular, rectification) by using a known method to remove a low-boiling fraction and a high-boiling fraction, thereby obtaining a mixture comprising 2-pentylhexadecanol and 2-heptyltetradecanol. The mixing ratio of the 2-pentylhexadecanol to the 2-heptyltetradecanol is preferably 20:80 to 70:30, more preferably 40:60 to 60:40, and particularly preferably 45:55 to 55:45, in terms of the weight ratio.

In step (III), the mixture obtained in step (II) is reacted with the compound represented by general formula (1a) (an esterification reaction) to obtain a mixture (lubricating base oil for a fluid bearing) comprising the compound represented by general formula (1) and the compound represented by general formula (2).

This reaction can be generally carried out in the presence of a solvent. Examples of solvents include aromatic hydrocarbon solvents, such as toluene and xylene; and the like.

Any catalyst that promotes the esterification reaction can be used. Examples of such catalysts include tin oxide, titanium tetraalkoxide, p-toluenesulfonic acid, and the like.

The amount of the compound represented by general formula (1a) is generally 1 to 1.1 moles, and preferably 1 to 1.05 moles, per mole of the mixture obtained in step (II).

This reaction can be generally carried out at 100 to 300° C. for 2 to 10 hours. After completion of the reaction, a post-treatment is performed by a known method to obtain a mixture (lubricating base oil for a fluid bearing) comprising the compound represented by general formula (1) and the compound represented by general formula (2).

The lubricating base oil for a fluid bearing is suitably used as a lubricating base oil for a fluid dynamic bearing or for an oil-impregnated sintered bearing.

The lubricating base oil for a fluid bearing may contain a base oil (additional base oil) other than the compound represented by general formula (1) and the compound represented by general formula (2). Examples of additional base oils include mineral oils (hydrocarbon oils obtained by purification of petroleum); poly-α-olefins; polybutenes; alkylbenzenes; alkylnaphthalenes; alicyclic hydrocarbon oils; iscomerized oils of synthetic hydrocarbons obtained by the Fischer-Tropsch process and like synthetic hydrocarbon oils; animal and vegetable oils; organic acid esters other than the compound represented by general formula (1) and the compound represented by general formula (2); polyalkylene glycols; ether-based base oils, such as polyvinyl ethers, polyphenyl ethers, and alkylphenyl ethers; and the like. At least one of these additional base oils may suitably be used.

Examples of mineral oils include solvent-refined mineral oils, mineral oils treated by hydrogenation, and wax isomerized oils; and usable mineral oils are those having a kinematic viscosity in the range of generally 1 to 25 mm²/s, and preferably 2 to 20 mm²/s at 100° C.

Examples of poly-α-olefins include polymers or copolymers of α-olefins having 2 to 16 carbon atoms (for example, ethylene, propylene, 1-butene, 1-hexene, 1-octene, 1-decene, 1-dodecene, 1-tetradecene, 1-hexadecene, etc.), the polymers or copolymers having a kinematic viscosity of 1 to 25 mm²/s at 100° C. and a viscosity index of 100 or more, and preferably a kinematic viscosity of 1.5 to 20 mm²/s at 100° C. and a viscosity index of 120 or more.

Examples of polybutenes include those obtained by polymerizing isobutylene, and those obtained by copolymerizing isobutylene with normal butylene; and those having a kinematic viscosity in the wide range of 2 to 40 mm²/s at 100° C. are generally usable.

Examples of alkylbenzenes include benzenes substituted with one or more linear or branched C₁-C₄₀ alkyl groups, such as monoalkylbenzenes, dialkylbenzenes, trialkylbenzenes, and tetraalkylbenzenes that have a molecular weight of 200 to 450.

Examples of alkylnaphthalenes include naphthalenes substituted with one or more linear or branched C₁-C₃₀ alkyl groups, such as monoalkylnaphthalenes and dialkylnaphthalenes.

Examples of animal and vegetable oils include beef tallow, lard, palm oil, coconut oil, rapeseed oil, castor oil, sunflower oil, and the like.

Examples of organic acid esters other than the compound represented by general formula (1) and the compound represented by general formula (2) include fatty acid monoesters (excluding the compound represented by general formula (1) and the compound represented by general formula (2)), aliphatic dibasic acid diesters, polyol esters, and other esters.

Examples of fatty acid monoesters (excluding the compound represented by general formula (1) and the compound represented by general formula (2)) include esters of a C₅-C₂₂ aliphatic linear or branched monocarboxylic acid and a C₃-C₂₂ linear or branched saturated or unsaturated aliphatic alcohol.

Examples of aliphatic dibasic acid diesters include diesters of a C₃-C₂ linear or branched saturated or unsaturated aliphatic alcohol with an aliphatic dibasic acid such as oxalic acid, malonic acid, succinic acid, glutaric acid, adipic acid, pimelic acid, suberic acid, azelaic acid, sebacic acid, 1,9-nonamethylenedicarboxylic acid, or 1,10-decamethylenedicarboxylic acid; or an anhydride thereof.

For polyol esters, it is possible to use full-esters of a polyol that has a neopentyl structure or a polyol that has a non-neopentyl structure with a C₃-C₂₂ linear or branched saturated or unsaturated aliphatic monocarboxylic acid. Examples of polyols that have a neopentyl structure include neopentyl glycol, 2,2-diethylpropanediol, 2-butyl-2-ethylpropanediol, trimethylolethane, trimethylolpropane, pentaerythritol, ditrimethylolpropane, dipentaerythritol, and the like. Examples of polyols that have a non-neopentyl structure include 1,3-propanediol, 1,4-butanediol, 1,5-pentanediol, 1,6-hexanediol, 1,7-heptanediol, 1,8-octanediol, 1,9-nonanediol, 1,10-decanediol, 1,2-propanediol, 2-methyl-1,3-propanediol, 1,3-butanediol, 2-methyl-1,4-butanediol, 1,4-pentanediol, 2-methyl-1,5-pentanediol, 3-methyl-1,5-pentanediol, 1,5-hexanediol, 2-methyl-1,6-hexanediol, 3-methyl-1,6-hexanediol, 1,6-heptanediol, 2-methyl-1,7-heptanediol, 3-methyl-1,7-heptanediol, 4-methyl-1,7-heptanediol, 1,7-octanediol, 2-methyl-1,8-octanediol, 3-methyl-1,8-octanediol, 4-methyl-1,8-octanediol, 1,8-nonanediol, 2-methyl-1,9-nonanediol, 3-methyl-1,9-nonanediol, 4-methyl-1,9-nonanediol, 5-methyl-1,9-nonanediol, 2-ethyl-1,3-hexanediol, glycerol, polyglycerol, sorbitol, and the like.

Examples of other esters include esters of a polymerized fatty acid such as dimer acid or hydrogenated dimer acid, or a hydroxy fatty acid such as a condensed castor oil fatty acid or a hydrogenated condensed castor oil fatty acid, with a C₃-C₂₂ linear or branched saturated or unsaturated aliphatic alcohol.

Examples of polyalkylene glycols include a polymer prepared from an alcohol and one or more C₂-C₄ linear or branched alkylene oxides by ring-opening polymerization. Examples of alkylene oxides include ethylene oxide, propylene oxide, and butylene oxide; it is possible to use polymers prepared from one of these, or copolymers prepared from a mixture of two or more of these. It is also possible to use such polyalkylene glycols wherein the hydroxy group(s) at one or both ends are etherified or esterified. The kinematic viscosity of the polymer is 5 to 1,000 mm²/s (40° C.), and preferably 5 to 500 mm²/s (40° C.).

Polyvinyl ethers are, for example, compounds obtained by polymerizing a vinyl ether monomer. Examples of monomers include methyl vinyl ether, ethyl vinyl ether, isopropyl vinyl ether, n-butyl vinyl ether, isobutyl vinyl ether, sec-butyl vinyl ether, tert-butyl vinyl ether, n-pentyl vinyl ether, n-hexyl vinyl ether, 2-methoxyethyl vinyl ether, 2-ethoxyethyl vinyl ether, and the like. The kinematic viscosity of the polymer is 5 to 1,000 mm²/s (40° C.), and preferably 5 to 500 mm²/s (40° C.).

Examples of polyphenyl ethers include compounds having a structure wherein the meta positions of two or more aromatic rings are connected by one or more ether linkages or thioether linkages; specifically, for example, bis(m-phenoxyphenyl)ether, m-bis(m-phenoxyphenoxy)benzene, and thioethers wherein one or more oxygen atoms thereof are replaced by one or more sulfur atoms.

Examples of alkylphenyl ethers include compounds wherein a polyphenyl ether is substituted with one or more linear or branched C₆-C₁₆ alkyl groups; in particular, alkyldiphenyl ethers containing one or more alkyl groups are preferable.

The lubricating base oil for a fluid bearing comprises the compound represented by general formula (1) and the compound represented by general formula (2), and may comprise one or more additional base oils, as necessary. It is preferred that the lubricating base oil for a fluid bearing consist essentially of the compound represented by general formula (1) and the compound represented by general formula (2). It is more preferred that the lubricating base oil for a fluid bearing consist of the compound represented by general formula (1) and the compound represented by general formula (2).

It is recommended that the content of the additional base oil in the lubricating base oil for a fluid bearing be generally 10 wt % or less; in order to improve the balance of physical properties, the content of the additional base oil in the lubricating base oil for a fluid bearing is more preferably 5 wt % or less.

### 2. Lubricating Oil Composition for Fluid Bearing

The lubricating oil composition for a fluid bearing of the present invention comprises the lubricating base oil for a fluid bearing described above. In order to improve the performance of the lubricating base oil for a fluid bearing, the composition may comprise, in addition to the lubricating base oil for a fluid bearing, one or more additives (e.g., antioxidants).

Examples of antioxidants include phenolic antioxidants, amine-based antioxidants, and the like. Among these, phenolic antioxidants and amine-based antioxidants are preferable.

As phenolic antioxidants, various known antioxidants used in the art can be used, without any particular limitation. Among these phenolic antioxidants, those containing preferably 6 to 100 carbon atoms in total, and more preferably 20 to 80 carbon atoms in total, are preferable.

Specific examples include 2,6-di-tert-butylphenol, 2,6-di-tert-butyl-p-cresol, 4,4′-methylenebis(2,6-di-tert-butylphenol), 4,4′-butylidenebis(3-methyl-6-tert-butylphenol), 2,2′-methylenebis(4-ethyl-6-tert-butylphenol), 2,2′-methylenebis(4-methyl-6-tert-butylphenol), 4,4′-isopropyl idenebisphenol, 2,4-dimethyl-6-tert-butylphenol, tetrakis[methylene-3-(3,5-di-tert-butyl-4-hydroxyphenyl) propionate]methane, 1,1,3-tris(2-methyl-4-hydroxy-5-tert-butylphenyl)butane, 1,3,5-trimethyl-2,4,6-tris(3,5-di-tert-butyl-4-hydroxybenzyl)benzene, 2,2′-dihydroxy-3,3′-di(α-methylcyclohexyl)-5,5′-dimethyl-diphenylmethane, 2,2′-isobutylidenebis(4,6-dimethylphenol), 2,6-bis(2′-hydroxy-3′-tert-butyl-5′-methylbenzyl)-4-methylphenol, 1,1′-bis(4-hydroxyphenyl)cyclohexane, 2,5-di-tert-amylhydroquinone, 2,5-di-tert-butylhydroquinone, 1,4-dihydroxyanthraquinone, 3-tert-butyl-4-hydroxyanisole, 2-tert-butyl-4-hydroxyanisole, 2,4-dibenzoylresorcinol, 4-tert-butylcatechol, 2,6-di-tert-butyl-4-ethylphenol, 2-hydroxy-4-methoxybenzophenone, 2,4-dihydroxybenzophenone, 2,2′-dihydroxy-4-methoxybenzophenone, 2,4,5-trihydroxybenzophenone, α-tocopherol, bis[2-(2-hydroxy-5-methyl-3-tert-butylbenzyl)-4-methyl-6-tert-butylphenyl]terephthalate, triethyleneglycol bis[3-(3-tert-butyl-5-methyl-4-hydroxyphenyl) propionate], 1,6-hexanediol-bis[3-(3,5-di-tert-butyl-4-hydroxyphenyl)propionate], and the like. Among these, 2,6-di-tert-butylphenol, 2,6-di-tert-butyl-p-cresol, 4,4′-methylenebis(2,6-di-tert-butylphenol), 4,4′-butylidenebis(3-methyl-6-tert-butylphenol), 2,2′-methylenebis(4-ethyl-6-tert-butylphenol), 2,2′-methylenebis (4-methyl-6-tert-butylphenol), 4,4′-isopropylidenebisphenol, 2,4-dimethyl-6-tert-butylphenol, tetrakis[methylene-3-(3,5-di-tert-butyl-4-hydroxyphenyl) propionate]methane, 1,1,3-tris(2-methyl-4-hydroxy-5-tert-butylphenyl) butane, 1,3,5-trimethyl-2,4,6-tris(3,5-di-tert-butyl-4-hydroxybenzyl)benzene, 2,6-di-tert-butyl-4-ethylphenol, bis[2-(2-hydroxy-5-methyl-3-tert-butylbenzyl)-4-methyl-6-tert-butylphenyl]terephthalate, triethyleneglycol bis[3-(3-tert-butyl-5-methyl-4-hydroxyphenyl) propionate], and 1,6-hexanediol-bis[3-(3,5-di-tert-butyl-4-hydroxyphenyl) propionate] are particularly preferable. 2,6-di-tert-butyl-p-cresol, 4,4′-methylenebis(2,6-di-tert-butylphenol), and 2,6-di-tert-butyl-4-ethylphenol are most preferable.

These phenolic antioxidants may be used singly, or in a combination of two or more. The amount of phenolic antioxidant is generally 0.01 to 5 parts by weight, and preferably 0.1 to 2 parts by weight, per 100 parts by weight of the lubricating base oil for a fluid bearing.

As amine-based antioxidants, various known antioxidants used in the art can be used without any particular limitation. Among these amine-based antioxidants, those containing preferably 6 to 60 carbon atoms in total, and more preferably 20 to 40 carbon atoms in total, are preferable.

Specific examples include diphenylamine compounds such as diphenylamine, monobutyldiphenylamine, monopentyldiphenylamine, monohexyl (including linear and branched) diphenylamine, monoheptyldiphenylamine, monooctyldiphenylamine, and like monoalkyldiphenylamines, in particular, mono(C₄-C₉ alkyl)diphenylamines (i.e., diphenylamines wherein one of the two benzene rings is mono-substituted with an alkyl group, in particular, a C₄-C₉ alkyl group, i.e., monoalkyl-substituted diphenylamines), p,p′-dibutyldiphenylamine, p,p′-dipentyldiphenylamine, p,p′-dihexyldiphenylamine, p,p′-diheptyldiphenylamine, p,p′-dioctyldiphenylamine, p,p′-dinonyldiphenylamine, and like di(alkylphenyl)amines, in particular, p,p′-di(C₄-C₉ alkylphenyl)amines (i.e., dialkyl-substituted diphenylamines wherein each of the two benzene rings is mono-substituted with an alkyl group, in particular, a C₄-C₉ alkyl group, and the two alkyl groups are identical), di(mono C₄-C₉ alkylphenyl)amines wherein the alkyl group on one of the benzene rings is different from the alkyl group on the other of the benzene rings, di(di-C₄-C₉ alkylphenyl)amines wherein at least one of the four alkyl groups on the two benzene rings is different from the rest of the alkyl groups; naphthylamine compounds such as N-phenyl-1-naphthylamine, N-phenyl-2-naphthylamine, 4-octylphenyl-1-naphthylamine, and 4-octylphenyl-2-naphthylamine; phenylenediamine compounds such as p-phenylenediamine, N-phenyl-N′-isopropyl-p-phenylenediamine, and N-phenyl-N′-(1,3-dimethylbutyl)-p-phenylenediamine; and the like. Among these, p,p′-dioctyldiphenylamine, p,p′-dinonyldiphenylamine, and N-phenyl-1-naphthylamine are particularly preferable.

In the present specification and claims, examples of alkyl include linear or branched C₁-C₂₀ alkyl, and preferable examples of alkyl include linear or branched C₁-C₁₂ alkyl. When there are a plurality of alkyl groups in the same molecule, the alkyl groups may be the same or different. When there are a plurality of alkyl groups having the same number of carbon atoms in the same molecule, the alkyl groups may be linear or branched.

The amine-based antioxidants may be used singly, or in a combination of two or more. The amount of amine-based antioxidant is generally 0.01 to 5 parts by weight, and preferably 0.1 to 2 parts by weight, per 100 parts by weight of the lubricating base oil for a fluid bearing.

One or more phenolic antioxidants and one or more amine-based antioxidants may be suitably used in combination. The ratio thereof is not particularly limited, and may be suitably selected from a wide range. The phenolic antioxidant(s) and the amine-based antioxidant (s) are preferably used in combination so that the weight ratio of phenolic antioxidant (I) to amine-based antioxidant (II), i.e., (I):(II), is 1:0.05-20, and particularly 1:0.2-5.

Examples of preferable combinations include a combination of one or more members selected from the group consisting of 2,6-di-tert-butyl-p-cresol, 4,4′-methylenebis(2,6-di-tert-butylphenol), and 2,6-di-tert-butyl-4-ethylphenol, with one or more members selected from the group consisting of p,p′-dioctyldiphenylamine, p,p′-dinonyldiphenylamine, and N-phenyl-1-naphthylamine.

Specific examples of preferable combinations include a combination of 2,6-di-tert-butyl-p-cresol and p,p′-dioctyldiphenylamine, a combination of 2,6-di-tert-butyl-p-cresol and p,p′-dinonyldiphenylamine, a combination of 2,6-di-tert-butyl-p-cresol and N-phenyl-1-naphthylamine, a combination of 4,4′-methylenebis(2,6-di-tert-butylphenol) and p,p′-dioctyldiphenylamine, a combination of 4,4′-methylenebis(2,6-di-tert-butylphenol) and p,p′-dinonyldiphenylamine, a combination of 4,4′-methylenebis(2,6-di-tert-butylphenol) and N-phenyl-1-naphthylamine, a combination of 2,6-di-tert-butyl-4-ethylphenol and p,p′-dioctyldiphenylamine, a combination of 2,6-di-tert-butyl-4-ethylphenol and p,p′-dinonyldiphenylamine, a combination of 2,6-di-tert-butyl-4-ethylphenol and N-phenyl-1-naphthylamine, and the like. Among these, for example, a combination of 4,4′-methylenebis(2,6-di-tert-butylphenol) and p,p′-dioctyldiphenylamine, a combination of 4,4′-methylenebis(2,6-di-tert-butylphenol) and p,p′-dinonyldiphenylamine, and a combination of 4,4′-methylenebis(2,6-di-tert-butylphenol) and N-phenyl-1-naphthylamine are recommended as more effective combinations in terms of excellent heat resistance.

When one or more phenolic antioxidants and one or more amine-based antioxidants are used in combination, the total amount of the phenolic antioxidant (s) and the amine-based antioxidant(s) is generally 0.01 to 5 parts by weight, and preferably 0.1 to 2 parts by weight, per 100 parts by weight of the lubricating base oil for a fluid bearing.

Incorporating an antioxidant described above into the lubricating base oil for a fluid bearing suppresses, for example, decomposition of the lubricating base oil in the presence of air, thus improving the heat resistance of the lubricating oil composition for a fluid bearing.

In order to further improve the performance of the lubricating oil composition for a fluid bearing, the lubricating oil composition for a fluid bearing may suitably contain at least one additive selected from the group consisting of metal detergents, ashless dispersants, oiliness agents, antiwear agents, extreme-pressure agents, metal deactivators, rust inhibitors, viscosity index improvers, pour point depressants, hydrolysis inhibitors, and the like. The amounts of such additives are not particularly limited as long as the effect of the present invention is ensured, and specific examples are as described below.

Examples of usable metal detergents include Ca-petroleum sulfonate, overbased Ca-petroleum sulfonate, Ca-alkylbenzene sulfonate, overbased Ca-alkylbenzene sulfonate, Ba-alkylbenzene sulfonate, overbased Ba-alkylbenzene sulfonate, Mg-alkylbenzene sulfonate, overbased Mg-alkylbenzene sulfonate, Na-alkylbenzene sulfonate, overbased Na-alkylbenzene sulfonate, Ca-alkylnaphthalene sulfonate, overbased Ca-alkylnaphthalene sulfonate, and like metal sulfonates; Ca-phenate, overbased Ca-phenate, Ba-phenate, overbased Ba-phenate, and like metal phenates; Ca-salicylate, overbased Ca-salicylate and like metal salicylates; Ca-phosphonate, overbased Ca-phosphonate, Ba-phosphonate, overbased Ba-phosphonate, and like metal phosphonates; overbased Ca-carboxylates; and the like. When such a metal detergent is used, the amount thereof may be generally 1 to 10 parts by weight, and preferably 2 to 7 parts by weight, per 100 parts by weight of the lubricating base oil for a fluid bearing.

Examples of ashless dispersants include polyalkenyl succinimides, polyalkenyl succinamides, polyalkenyl benzylamines, polyalkenyl succinic acid esters, and the like. These ashless dispersants may be used singly, or in combination. When such an ashless dispersant is used, the amount thereof may be generally 1 to 10 parts by weight, and preferably 2 to 7 parts by weight, per 100 parts by weight of the lubricating base oil for a fluid bearing.

Examples of oiliness agents include stearic acid, oleic acid, and like saturated or unsaturated aliphatic monocarboxylic acids; dimer acid, hydrogenated dimer acid, and like polymerized fatty acids; ricinoleic acid, 12-hydroxystearic acid, and like hydroxyfatty acids; lauryl alcohol, oleyl alcohol, and like saturated or unsaturated aliphatic monoalcohols; stearyl amine, oleyl amine, and like saturated or unsaturated aliphatic monoamines; lauramide, oleamide, and like saturated or unsaturated aliphatic monocarboxylic acid amides; batyl alcohol, chimyl alcohol, selachyl alcohol, and like glycerin ethers; lauryl polyglycerol ether, oleyl polyglyceryl ether, and like alkyl or alkenyl polyglyceryl ethers; di(2-ethylhexyl)monoethanolamine, diisotridecyl monoethanolamine, and like poly(alkylene oxide) adducts of alkyl or alkenylamine; and the like. These oiliness agents may be used singly, or in combination. When such an oiliness agent is used, the amount thereof may be generally 0.01 to 5 parts by weight, and preferably 0.1 to 3 parts by weight, per 100 parts by weight of the lubricating base oil for a fluid bearing.

Examples of antiwear agents and extreme-pressure agents include phosphorus-based compounds such as tricresyl phosphate, cresyldiphenyl phosphate, alkylphenyl phosphates, tributyl phosphate, dibutyl phosphate, and like phosphoric acid esters, tributyl phosphite, dibutyl phosphite, triisopropyl phosphite and like phosphorous acid esters, as well as amine salts thereof; sulfur-based compounds such as sulfurized oils and fats, sulfurized oleic acid and like sulfurized fatty acids, di-benzyl disulfide, sulfurized olefins, and dialkyl disulfides; organometallic compounds such as Zn-dialkyldithio phosphates, Mo-dialkyldithio phosphates, and Mo-dialkyldithio carbamates; and the like. These antiwear agents may be used singly, or in combination. When such an antiwear agent is used, the amount thereof may be generally 0.01 to 10 parts by weight, and preferably 0.1 to 5 parts by weight, per 100 parts by weight of the lubricating base oil for a fluid bearing.

Examples of metal deactivators include benzotriazole-based compounds, thiadiazole-based compounds, gallic acid ester-based compounds, and the like. These metal deactivators may be used singly, or in combination. When such a metal deactivator is used, the amount thereof may be generally 0.01 to 0.4 parts by weight, and preferably 0.01 to 0.2 parts by weight, per 100 parts by weight of the lubricating base oil for a fluid bearing. Examples of rust inhibitors include dodecenylsuccinic acid half-esters, octadecenylsuccinic anhydride, dodecenylsuccinic amide, and like alkyl or alkenyl succinic acid derivatives; sorbitan monooleate, glycerol monooleate, pentaerythritol monooleate, and like partial esters of polyhydric alcohols; Ca-petroleum sulfonate, Ca-alkylbenzene sulfonate, Ba-alkylbenzene sulfonate, Mg-alkylbenzene sulfonate, Na-alkylbenzene sulfonate, Zn-alkylbenzene sulfonate, Ca-alkylnaphthalene sulfonate, and like metal sulfonates; rosin amine, N-oleyl sarcosine, and like amines; dialkyl phosphite amine salts; and the like. These rust inhibitors may be used singly, or in combination. When such a rust inhibitor is used, the amount thereof may be generally 0.01 to 5 parts by weight, and preferably 0.05 to 2 parts by weight, per 100 parts by weight of the lubricating base oil for a fluid bearing.

Examples of viscosity index improvers include polyalkylmethacrylates, polyalkylstyrenes, polybutenes, ethylene-propylene copolymers, styrene-diene copolymers, styrene-maleic anhydride ester copolymers, and like olefin copolymers. These viscosity index improvers may be used singly, or in combination. When such a viscosity index improver is used, the amount thereof may be generally 0.1 to 15 parts by weight, preferably 0.5 to 7 parts by weight, per 100 parts by weight of the lubricating base oil for a fluid bearing.

Examples of pour point depressants include condensates of chlorinated paraffin and alkylnaphthalene; condensates of chlorinated paraffin and phenol; and polyalkylmethacrylates, polyalkylstyrenes, polybutenes, etc., which are also viscosity index improvers as mentioned above. These pour point depressants may be used singly, or in combination. When such a pour point depressant is used, the amount thereof may be generally 0.01 to 5 parts by weight, and preferably 0.1 to 3 parts by weight, per 100 parts by weight of the lubricating base oil for a fluid bearing.

Examples of usable hydrolysis inhibitors include alkyl glycidyl ethers, alkyl glycidyl esters, alkylene glycol glycidyl ethers, alicyclic epoxides, phenyl glycidyl ether and like epoxy compounds; and di-tert-butylcarbodiimide, 1,3-di-p-tolylcarbodiimide, and like carbodiimide compounds. The amount thereof may be generally 0.05 to 2 parts by weight per 100 parts by weight of the lubricating base oil for a fluid bearing.

### 3. Fluid Bearing

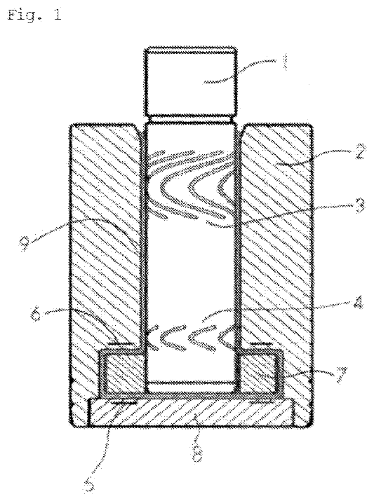

The present invention also provides a fluid bearing comprising the lubricating oil composition for a fluid bearing described above. Specific examples of the fluid bearing of the present invention include the fluid bearing shown in FIG. 1. FIG. 1 is an example of a cross-sectional view schematically showing an outline configuration of the fluid bearing of the present invention.

The fluid bearing of the present invention does not have a mechanism such as a ball bearing, and includes a shaft and a sleeve. The spacing between the shaft and the sleeve is maintained by a lubricating oil composition accommodated between them so that the shaft and the sleeve do not come into direct contact with each other. There is no particular limitation on the bearing mechanically, as long as it is such a bearing. The fluid bearing shown in FIG. 1 is an example of a fluid bearing in which a shaft (1) is provided with radial dynamic pressure-generating grooves (3) and (4), and thrust dynamic pressure-generating grooves (5) and (6) are provided above and below a thrust plate (7). In this example, these dynamic pressure grooves (3), (4), (5), and (6) are formed in a herringbone shape; however, the shape of the dynamic pressure grooves (3), (4), (5), and (6) is not limited thereto. The dynamic pressure grooves (3), (4), (5), and (6) may be formed in the shape of, for example, a spiral, an arc, or a straight line.

The radial dynamic pressure-generating grooves (3) and (4) may be formed on the inner peripheral surface of a sleeve (2) instead of the outer peripheral surface of the shaft (1). The thrust dynamic pressure-generating grooves (5) and (6) may be formed on the upper surface and the lower surface of the thrust plate (7), instead of the lower end surface of the sleeve (2) and the upper surface of a counter plate (8). The lubricating oil composition (9) of the present invention is enclosed in minute gaps between these dynamic pressure grooves (3), (4), (5), and (6), and their respective opposing surfaces.

In the fluid bearing having the above configuration, for example, when the shaft (1) is rotated, dynamic pressure in the radial direction is generated in the lubricating oil composition in the minute gaps by the dynamic pressure grooves (3) and (4), and dynamic pressure in the axial direction (thrust force) is also generated in the lubricating oil composition in the minute gaps by the bearing surface; these dynamic pressures cause the shaft (1) with the thrust plate (7) to rotate at high speed in a state in which it is not in contact with the sleeve (2) and counter plate (8).

The fluid bearing of the present invention uses as the lubricating oil (9) the lubricating oil composition for a bearing, which contains the lubricating base oil for a fluid bearing having good stability, viscosity properties, low-temperature properties, and volatility resistance; thus, a bearing life longer than that of a fluid bearing using a conventional lubricating oil composition can be obtained, without increasing the amount of the lubricating oil composition retained. The fluid bearing of the present invention is thus suitable as a fluid bearing applied to, for example, spindle motors, which are required to be compact, have high accuracy, and rotate at high speed.

### 4. Spindle Motor

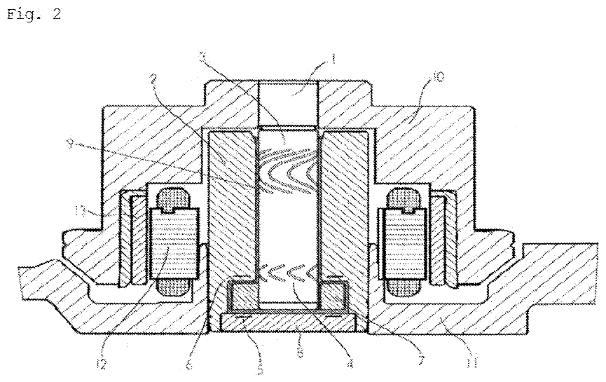

The present invention also provides a spindle motor comprising the fluid bearing described above. Specific examples of the spindle motor of the present invention include the spindle motor shown in FIG. 2. FIG. 2 is an example of a cross-sectional view schematically showing an outline configuration of the spindle motor of the present invention.

In the spindle motor of the present invention, a motor drive unit is configured such that stator coil (12) are provided on a wall formed on a base (11), and such that a rotor magnet (13) is attached on the inner peripheral surface of a hub (10) so as to face the stator coil (12). When the rotating part is rotated by the motor drive unit, dynamic pressure is generated in both the radial direction and the thrust direction in the lubricating oil composition (9), thus maintaining the rotation in a state in which the rotating part and the fixed part are not in contact with each other.

### EXAMPLES

The present invention is described below in detail with reference to Examples; however, the present invention is not limited to these Examples. The physical properties and chemical properties of the lubricating base oils and lubricating oil compositions in the Examples were evaluated by the following methods. Reagents were used as compounds that are not specified here.

### Compounds

### Raw Materials

1-tetradecanol: “Conol 1495” (produced by New Japan Chemical Co., Ltd.)

1-heptanol (produced by Tokyo Chemical Industry Co., Ltd.)

n-octanoic acid: “Caprylic Acid” (produced by New Japan Chemical Co., Ltd.)

n-nonanoic acid: “Nonanoic Acid” (produced by Tokyo Chemical Industry Co., Ltd.)

n-decanoic acid: “Capric Acid” (produced by New Japan Chemical Co., Ltd.)

n-undecanoic acid: “Undecanoic Acid” (produced by Tokyo Chemical Industry Co., Ltd.)

n-dodecanoic acid: “Lauric Acid P” (produced by New Japan Chemical Co., Ltd.)

n-tetradecanoic acid: “Myristic Acid” (produced by New Japan Chemical Co., Ltd.)

### Antioxidants

Amine-based antioxidant
p,p′-dioctyl (including linear and branched) diphenylamine: “bis(4-octylphenyl)amine” (produced by Ark Pharm, Inc.; hereinafter referred to as “DODPA”)

Phenolic antioxidant
4,4′-methylenebis(2,6-di-tert-butylphenol) (produced by Tokyo Chemical Industry Co., Ltd.; hereinafter referred to as “MBDBP”)

### (a) Acid Value

Measurement was performed according to JIS-K-2501 (2003). The detection limit is 0.01 KOH mg/g.

### (b) Kinematic Viscosity

The kinematic viscosity at 40° C. and 100° C. was measured according to JIS-K-2283 (2000).

Evaluation of kinematic viscosity at 40° C.
A: 11 mm²/s or more, and less than 16 mm²/s
B: 8 mm²/s or more, and less than 11 mm²/s; or 16 mm²/s or more, and less than 20 mm²/s
C: 6 mm²/s or more, and less than 8 mm²/s; or 20 mm²/s or more, and less than 25 mm²/s
D: less than 6 mm²/s, or 25 mm²/s or more

### (c) Viscosity Index

The viscosity index was calculated according to JIS-K-2283 (2000).

### Evaluation of Viscosity Index

A: 155 or more
B: 145 or more, and less than 155
C: 120 or more, and less than 145
D: less than 120

### (d) Low-Temperature Fluidity Test (Pour Point)

The pour point was measured according to JIS-K-2269 (1987).

### Evaluation of Low-Temperature Fluidity

A: −12.5° C. or less
B: more than −12.5° C., and −7.5° C. or less
C: more than −7.5° C., and 0° C. or less
D: more than 0° C.

### (e) Evaporation Resistance

About 10 mg of the lubricating base oil for a fluid bearing or the lubricating oil composition for a fluid bearing was accurately weighed (to the third decimal place), and placed in a TG-DTA device (produced by SII NanoTechnology Inc., device name: EXSTAR 6000 series, T3/DTA6200). The temperature at which the weight was reduced by 5% from the initial weight (temperature at which the weight was reduced by 5%) under the following measurement conditions was used as an index of evaporation resistance.

### Measurement Conditions

Temperature increase rate: 10° C./min
Amount of nitrogen that is allowed to flow: 200 ml/min Measurement start temperature: 50° C.
Evaluation of evaporation resistance (temperature at which the weight was reduced by 5%)
A: 275° C. or more
B: 270° C. or more, and less than 275° C.
C: 265° C. or more, and less than 270° C.
D: less than 265° C.

### (f) Hydrolytic Stability Test

The hydrolysis test was performed in the following manner. 2 g of the lubricating base oil for a fluid bearing or the lubricating oil composition for a fluid bearing, and 0.2 g of ion-exchanged water were weighed into a test tube, and the test tube was sealed while being freeze-degassed. After the sealed test tube was allowed to stand in a Fine Oven at 160° C. for 24 hours, the test liquid was removed from the test tube. The test liquid was allowed to stand, and an oil layer was removed by separation. The acid value of the oil layer was then measured. As an index of hydrolytic stability, an increase in acid value between before and after the hydrolysis test was calculated.

### Evaluation of Hydrolytic Stability (Increase in Acid Value)

A: 0.25 KOH mg/g or less
B: more than 0.25 KOH mg/g, and 0.50 KOH mg/g or less
C: more than 0.50 KOH mg/g, and 0.80 KOH mg/g or less
D: more than 0.80 KOH mg/g

### (g) Evaluation of Lubricating Base Oil for Fluid Bearing

The lubricating base oil for a fluid bearing and the lubricating oil composition for a fluid bearing were evaluated as follows. In the results of evaluation of kinematic viscosity at 40° C., evaluation of viscosity index, evaluation of low-temperature fluidity, evaluation of evaporation resistance, and evaluation of hydrolytic stability, if the number of Cs or Ds was 1 or more, the oil or the composition was evaluated as unsuitable; if the number of Bs was 2 or less (it was rated as A in other evaluations), the oil or the composition was evaluated as good; and if the number of Be was 1 or less (it was rated as A in other evaluations), the oil or the composition was evaluated as particularly good.

### Production Example 1

Referring to JPS49-035308A, an alcohol dimerization reaction was performed using 1-tetradecanol and 1-heptanol. Specifically, 9.34 moles of 1-tetradecanol, 9.34 moles of 1-heptanol, 10.3 g of a 50% aqueous potassium hydroxide solution, 0.3 g of a copper chromium catalyst, and 1.5 g of activated carbon were added, and a dimerization reaction was carried out at 250° C. After the reaction, the copper chromium catalyst, activated carbon, and carboxylic acid potassium salt were removed by filtration to obtain a crude product containing four dimerized alcohols, i.e., 2-pentylnonanol, 2-pentylhexadecanol, 2-heptyltetradecanol, and 2-dodecylhexadecanol.

The obtained crude dimerized alcohol product was subjected to rectification to collect 2-pentylnonanol as a prefraction, and 2-heptyltetradecanol and then 2-pentylhexadecanol as main fractions. The obtained 2-pentylhexadecanol and 2-heptyltetradecanol were mixed at a desired ratio, and the mixture was used as an ester raw material described below.

### Production Example 2

0.416 moles of the mixture of 2-heptyltetradecanol and 2-pentylhexadecanol (50:50) obtained in Production Example 1, 0.428 moles of n-nonanoic acid, xylene (10 wt % relative to the total amount of raw materials), and a tin oxide catalyst (0.05 wt % relative to the total amount of raw materials) as a catalyst were placed in a 500-ml four-necked flask equipped with a stirrer, a thermometer, and a water fraction receiver with a cooling pipe. After purging with nitrogen, the mixture was gradually heated to 220° C. An esterification reaction was performed while removing distilled water using the water fraction receiver with reference to the theoretical water amount (7.48 g), and adjusting the decompression degree so that reflux occurred. The reaction was performed until the theoretical amount of water was distilled.

After completion of the reaction, xylene and the remaining raw material n-nonanoic acid were removed by distillation to obtain a crude esterified product. After neutralization with 2 equivalents of a caustic soda aqueous solution relative to the acid value of the obtained crude esterified product, water-washing was repeated until the washing water became neutral. Furthermore, after the obtained crude esterified product was subjected to adsorption treatment with activated carbon, the activated carbon was removed by filtration, thereby obtaining a mixture of n-nonanoic acid (2-pentylhexadecyl) and n-nonanoic acid (2-heptyltetradecyl) (50:50).

The acid value was less than 0.01 KOR rrg/g. Hereinafter, the obtained 2-pentylhexadecyl n-nonanoate is referred to as “C5C16-C9,” and the obtained 2-heptyltetradecyl n-nonanoate is referred to as “C7C14-C9.”

### Production Example 3

A mixture of 2-pentylhexadecyl n-decanoate and 2-heptyltetradecyl n-decanoate (50:50) having an acid value of less than 0.01 KOH mg/g was obtained in the same manner as in Production Example 2, except that n-decanoic acid was used in place of n-nonanoic acid. Hereinafter, the obtained 2-pentyihexadecyl n-decanoate is referred to as “C5C16-C10,” and the obtained 2-heptyltetradecyl n-decanoate is referred to as “C7C14-C10.”

### Production Example 4

A mixture of 2-pentylhexadecyl n-undecanoate and 2-heptyltetradecyl n-undecanoate (50:50) having an acid value of less than 0.01 KOH mg/g was obtained in the same manner as in Production Example 2, except that n-undecanoic acid was used in place of n-nonanoic acid. Hereinafter, the obtained 2-pentylhexadecyl n-undecanoate is referred to as “C5C16-C11,” and the obtained 2-heptyltetradecyl n-undecanoate is referred to as “C7C14-C11.”

### Production Example 5

A mixture of 2-pentylhexadecyl n-decanoate and 2-heptyltetradecyl n-decanoate (40:60) having an acid value of less than 0.01 KOH mg/g was obtained in the same manner as in Production Example 2, except that n-decanoic acid was used in place of n-nonanoic acid; and that a mixture of 2-pentylhexadecanol and 2-heptyltetradecanol (40:60) was used. Hereinafter, the obtained 2-pentylhexadecyl n-decanoate is referred to as “C5C16-C10,” and the obtained 2-heptyltetradecyl n-decanoate is referred to as “C7C14-C10.”

### Production Example 6

A mixture of 2-pentylhexadecyl n-undecanoate and 2-heptyltetradecyl n-undecanoate (60:40) having an acid value of less than 0.01 KOH mg/g was obtained in the same manner as in Production Example 2, except that n-undecanoic acid was used in place of n-nonanoic acid; and that a mixture of 2-pentyihexadecanol and 2-heptyltetradecanol (60:40) was used. Hereinafter, the obtained 2-pentylhexadecyl n-undecanoate is referred to as “C5C16-C11,” and the obtained 2-heptyltetradecyl n-undecanoate is referred to as “C7C14-C11.”

### Production Example 7

A mixture of 2-pentylhexadecyl n-undecanoate and 2-heptyltetradecyl n-undecanoate (50:50) having an acid value of less than 0.01 KOH mg/g was obtained in the same manner as in Production Example 2, except that n-undecanoic acid was used in place of n-nonanoic acid.

### Production Example 8

A mixture of 2-pentylhexadecyl n-undecanoate and 2-heptyltetradecyl n-undecanoate (45:55) having an acid value of less than 0.01 KOH mg/g was obtained in the same manner as in Production Example 2, except that n-undecanoic acid was used in place of n-nonanoic acid; and that a mixture of 2-pentyihexadecanol and 2-heptyltetradecanol (45:55) was used.

### Production Example 9

A mixture of 2-pentylhexadecyl n-undecanoate and 2-heptyltetradecyl n-undecanoate (40:60) having an acid value of less than 0.01 KOH mg/g was obtained in the same manner as in Production Example 2, except that n-undecanoic acid was used in place of n-nonanoic acid; and that a mixture of 2-pentylhexadecanol and 2-heptyltetradecanol (40:60) was used.

### Production Example 10

A mixture of 2-pentylhexadecyl n-dodecanoate and 2-heptyltetradecyl n-dodecanoate (50:50) having an acid value of less than 0.01 KOH mg/g was obtained in the same manner as in Production Example 2, except that n-dodecanoic acid was used in place of n-nonanoic acid. Hereinafter, the obtained 2-pentylhexadecyl n-dodecanoate is referred to as “C5C16-C12,” and the obtained 2-heptyltetradecyl n-dodecanoate is referred to as “C7C14-C12.”

### Examples 1 to 9

The mixtures of the ester compounds obtained in Production Examples 2 to 10 were each evaluated as a lubricating base oil for a fluid bearing. The kinematic viscosity and the viscosity index of each base oil were measured; and a low-temperature fluidity test (pour point), an evaporation resistance test, and a hydrolytic stability test were performed. Table 1 shows the results.

TABLE 1

| | | | | | | | | Evaporation | Acid value | | |
|---|---|---|---|---|---|---|---|---|---|---|---|
| | | | | Kinematic | | | | resistance | | After | Increase |
| | Lubricating base oil for | | Ratio | viscosity | | Viscosity | Pour | 5% weight | | hydrolysis | (KOH |
| | fluid bearing | | (1):(2) | (mm²/s) | | index | point | reduction | Initial | test | mg/g) |
| | Compound | Compound | (Weight | 40° C. | | (Eval- | (° C.) | (° C.) | (KOH | (KOH | (Eval- |
| | (1) | (2) | ratio) | (Evaluation) | 100° C. | uation) | (Evaluation) | (Evaluation) | mg/g) | mg/g) | uation) |
| Example 1 | C5C16-C9 | C7C14-C9 | 50.50 | 12.8 | 3.44 | 147 | −22.5 | 278 | ≤0.01 | 0.19 | 0.18 |
| (Production | | | | (A) | | (B) | (A) | (A) | | | (A) |
| Example 2) | | | | | | | | | | | |
| Example 2 | C5C16-C10 | C7C14-C10 | 50.50 | 14.1 | 3.71 | 160 | −17.5 | 284 | ≤0.01 | 0.20 | 0.19 |
| (Production | | | | (A) | | (A) | (A) | (A) | | | (A) |
| Example 3) | | | | | | | | | | | |
| Example 3 | C5C16-C11 | C7C14-C11 | 50.50 | 14.0 | 3.67 | 156 | −17.5 | 283 | ≤0.01 | 0.20 | 0.19 |
| (Production | | | | (A) | | (A) | (A) | (A) | | | (A) |
| Example 4) | | | | | | | | | | | |
| Example 4 | C5C16-C10 | C7C14-C10 | 40.60 | 13.9 | 3.65 | 156 | −20 | 283 | ≤0.01 | 0.20 | 0.19 |
| (Production | | | | (A) | | (A) | (A) | (A) | | | (A) |
| Example 5) | | | | | | | | | | | |
| Example 5 | C5C16-C11 | C7C14-C11 | 60.40 | 15.3 | 3.95 | 165 | −15 | 287 | ≤0.01 | 0.19 | 0.18 |
| (Production | | | | (A) | | (A) | (A) | (A) | | | (A) |
| Example 6) | | | | | | | | | | | |
| Example 6 | C5C16-C11 | C7C14-C11 | 50.50 | 15.2 | 3.91 | 161 | −17.5 | 287 | ≤0.01 | 0.18 | 0.17 |
| (Production | | | | (A) | | (A) | (A) | (A) | | | (A) |
| Example 7) | | | | | | | | | | | |
| Example 7 | C5C16-C11 | C7C14-C11 | 45.55 | 15.2 | 3.89 | 160 | −20 | 288 | ≤0.01 | 0.18 | 0.17 |
| (Production | | | | (A) | | (A) | (A) | (A) | | | (A) |
| Example 8) | | | | | | | | | | | |
| Example 8 | C5C16-C11 | C7C14-C11 | 40.60 | 15.1 | 3.87 | 158 | −20 | 288 | ≤0.01 | 0.18 | 0.17 |
| (Production | | | | (A) | | (A) | (A) | (A) | | | (A) |
| Example 9) | | | | | | | | | | | |
| Example 9 | C5C16-C12 | C7C14-C12 | 50.50 | 16.3 | 4.12 | 164 | −12.5 | 293 | ≤0.01 | 0.18 | 0.17 |
| (Production | | | | (B) | | (A) | (A) | (A) | | | (A) |
| Example 10) | | | | | | | | | | | |

### Comparative Examples 1 to 6

As comparative examples, diisodecyl adipate (DIDA), di(2-ethylhexyl) sebacate (DOS), 2-octyldecyl n-decanoate (ester A), 2-decyltetradecyl n-decanoate (ester B), an ester of n-decanoic acid and trimethylolpropane (ester C), and an ester of n-dodecanoic acid and neopentylglycol (ester D) were each evaluated as a lubricating base oil for a fluid bearing that was not the present invention. The kinematic viscosity and the viscosity index of each base oil were measured; and a low-temperature fluidity test (pour point), an evaporation resistance test, and a hydrolytic stability test were performed. Table 2 shows the results.

TABLE 2

| | | | | | | Evaporation | Acid value | | |
|---|---|---|---|---|---|---|---|---|---|
| | | | | | | resistance | | After | |
| | | Kinematic viscosity | | | Pour | 5% weight | | hydrolysis | Increase |
| | Lubricating | (mm²/s) | | Viscosity | point | reduction | Initial | test | (KOH |
| | base oil for | 40° C. | | index | (° C.) | (° C.) | (KOH | (KOH | mg/g) |
| | fluid bearing | (Evaluation) | 100° C. | (Evaluation) | (Evaluation) | (Evaluation) | mg/g) | mg/g) | (Evaluation) |
| Comparative | DOS | 11.5 | 3.21 | 153 | <−60 | 262 | ≤0.01 | 1.10 | 1.09 |
| Example 1 | | (A) | | (B) | (A) | (D) | | | (D) |
| Comparative | DIDA | 14.5 | 3.50 | 148 | <−60 | 259 | ≤0.01 | 0.51 | 0.50 |
| Example 2 | | (A) | | (B) | (A) | (D) | | | (B) |
| Comparative | Ester A | 12.5 | 3.34 | 147 | −12.5 | 268 | ≤0.01 | 0.21 | 0.20 |
| Example 3 | | (A) | | (B) | (A) | (C) | | | (A) |
| Comparative | Ester B | 16.6 | 4.15 | 162 | 5 | 299 | ≤0.01 | 0.21 | 0.20 |
| Example 4 | | (B) | | (A) | (D) | (A) | | | (A) |
| Comparative | Ester C | 17.0 | 3.94 | 130 | −50 | 286 | 0.02 | 11.61 | 11.59 |
| Example 5 | | (B) | | (C) | (A) | (A) | | | (D) |
| Comparative | Ester D | 14.8 | 3.84 | 160 | 15 | 279 | ≤0.01 | 1.03 | 1.02 |
| Example 6 | | (A) | | (A) | (D) | (A) | | | (D) |

### Examples 10 to 12

Lubricating oil compositions for a fluid bearing of the present invention were prepared by adding 1 part by weight of one or more antioxidants to 100 parts by weight of the lubricating base oil for a fluid bearing of Example 6. The kinematic viscosity and the viscosity index of each of the prepared lubricating oil compositions were measured; and a low-temperature fluidity test (pour point), an evaporation resistance test, and a hydrolytic stability test were performed. Table 3 shows the results.

TABLE 3

| | | | | | | | Evaporation | Acid value | | |
|---|---|---|---|---|---|---|---|---|---|---|
| | | | | | | | resistance | | After | |
| | Lubricating | | Kinematic viscosity | | | Pour | 5% weight | | hydrolysis | Increase |
| | base oil for | Antioxidant | (mm²/s) | | Viscosity | point | reduction | Initial | test | (KOH |
| | fluid bearing | (Parts by | 40° C. | | index | (° C.) | (° C.) | (KOH | (KOH | mg/g) |
| | (Parts by weight) | weight) | (Evaluation) | 100° C. | (Evaluation) | (Evaluation) | (Evaluation) | mg/g) | mg/g) | (Evaluation) |
| Example 10 | Base oil of | DODPA | 15.3 | 3.91 | 159 | −17.5 | 287 | ≤0.01 | 0.20 | 0.19 |
| | Example 6 | (1) | (A) | | (A) | (A) | (A) | | | (A) |
| | (100) | | | | | | | | | |
| Example 11 | Base oil of | MBDBP | 15.3 | 3.91 | 159 | −17.5 | 287 | ≤0.01 | 0.19 | 0.18 |
| | Example 6 | (1) | (A) | | (A) | (A) | (A) | | | (A) |
| | (100) | | | | | | | | | |
| Example 11 | Base oil of | DODPA | 15.3 | 3.91 | 159 | −17.5 | 287 | ≤0.01 | 0.19 | 0.18 |
| | Example 6 | (0.5) | (A) | | (A) | (A) | (A) | | | (A) |
| | (100) | MBDBP | | | | | | | | |
| | | (0.5) | | | | | | | | |

Table 1 shows that the lubricating base oils for a fluid bearing of the present invention are excellent lubricating base oils having excellent hydrolysis resistance, a high viscosity index, and good low-temperature fluidity and evaporation resistance. Further, Table 3 shows that the lubricating oil compositions for a fluid bearing of the present invention are also excellent lubricating oil compositions having excellent hydrolysis resistance, a high viscosity index, and good low-temperature fluidity and evaporation resistance.

### INDUSTRIAL APPLICABILITY

The lubricating base oil for a fluid bearing of the present invention has excellent hydrolysis resistance and low-temperature fluidity, a high viscosity index, and good evaporation resistance; thus, a spindle motor with a fluid bearing can be used stably for a long period of time by using the base oil of the present invention as a lubricating base oil for a fluid bearing.

1. A lubricating base oil for a fluid bearing, the lubricating base oil comprising a compound represented by general formula (1): 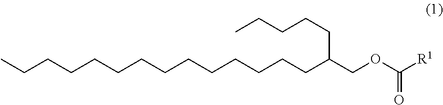
wherein R¹ represents a linear C₇-C₁₃ alkyl group, and a compound represented by general formula (2): 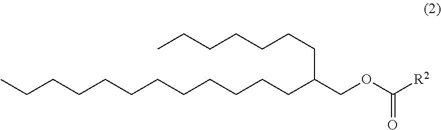
wherein R² represents a linear C₇-C₁₃ alkyl group.

2. The lubricating base oil for a fluid bearing according to claim 1, wherein R¹ in general formula (1) is a linear C₈-C₁₁ alkyl group, and R² in general formula (2) is a linear C₈-C₁₁ alkyl group.

3. The lubricating base oil for a fluid bearing according to claim 1, wherein R¹ in general formula (1) and R² in general formula (2) are the same linear alkyl group.

4. The lubricating base oil for a fluid bearing according to claim 1, wherein the weight ratio of the compound represented by general formula (1) to the compound represented by general formula (2) is 20:80 to 70:30.

5. The lubricating base oil for a fluid bearing according to claim 1, wherein the total amount of the compound represented by general formula (1) and the compound represented by general formula (2) in the lubricating base oil for a fluid bearing is 90 wt % or more.

6. The lubricating base oil for a fluid bearing according to claim 1, wherein the lubricating base oil for a fluid bearing is a lubricating base oil for a fluid dynamic bearing or a lubricating base oil for an oil-impregnated sintered bearing.

7. A lubricating oil composition for a fluid bearing, the composition comprising the lubricating base oil for a fluid bearing according to claim 1.

8. The lubricating oil composition for a fluid bearing according to claim 7, further comprising an antioxidant.

9. A fluid bearing comprising the lubricating oil composition for a fluid bearing according to claim 7.

10. A spindle motor comprising the fluid bearing according to claim 9.

11. A method for producing a mixture comprising a compound represented by general formula (1): 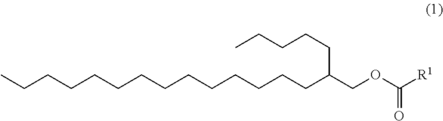
wherein R¹ represents a linear C₇-C₁₃ alkyl group; and a compound represented by general formula (2): 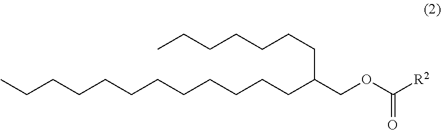
wherein R² represents a linear C₇-C₁₃ alkyl group,
R¹ in general formula (1) and R² in general formula (2) being the same linear C₈-C₁₁ alkyl group, the method comprising:
(I) subjecting 1-tetradecanol and 1-heptanol to a dimerization reaction to obtain a crude dimerized alcohol product,
(II) (a) distilling the obtained crude dimerized alcohol product to separately collect 2-pentylhexadecanol and 2-heptyltetradecanol, and mixing the 2-pentylhexadecanol and the 2-heptyltetradecanol at a predetermined ratio, thereby obtaining a mixture comprising the 2-pentylhexadecanol and the 2-heptyltetradecanol, or (b) distilling the obtained crude dimerized alcohol product to remove a low-boiling fraction and a high-boiling fraction, thereby obtaining a mixture comprising 2-pentylhexadecanol and 2-heptyltetradecanol; and
(III) reacting the obtained mixture with a compound represented by general formula (1a): 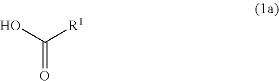
wherein R¹ is the same as above.
